SEQUENCE LISTING (2 sequences)

SEQ ID NO: 1 - DNA, 4540 bp, Homo sapiens
aaactgcgtttaccttaaacaggcttactgtgttcactgctggacgcctctgggcaatcttaccctcctgggaactgagagagacaatgctaggagccagaaacatgaggaatacatgagctcggcgccaatggaacatgctttcacaccgggtgcagctaaccaccacatcatacaaaactcgtcgctgacgttttagatgtataatctgattggcatggactcacagaggagggagtaagggaagagtaagaagatttcaaaggagagacacaggttgcaggttagcaggagaacaggcaagccaaatgcaaaggagccacttcagaaatgtgtcacagaaaagtgaaaatgcaacctagtggtaagtgaagaggggaagaagaaagaaaaaggaccagaaccgtgaactgaagggacagggaacagccagacgagagcttcagccatcacgaggatgatttcggaacctggagaaaatgtaagttaaatatatctacactctgatcctatctcaagagagagatattttactcatttcctggttgtgaatgatgggctcttggaagcactgtctttttagcgcgtctcttatctctgccctgatttttgtatttgtttacaatactgagttatgggagaataaacgttttctgagggcagctctgtccaatgcttcactgttagcagaagcctgtcatcagatttttgaggggaaagttttttacccaacagaaaatgcattgaaaactacccttgatgaagctacctgctatgagtacatggttcgaagccactatgtaacagaaacactctctgaagaagaggctgggttccctttagcttacacagtgaccatccacaaagacttcggcacttttgagaggctcttcagggcgatttatatgccccaaaatgtctactgtgtgcacctggatcagaaggcgacggatgcctttaaaggtgcagtgaaacagttactcagctgcttcccaaatgcttttctggcttccaagaaggagtcggttgtctatggggggatctccaggctccaggctgacctgaactgcctggaagaccttgtggcctctgaagttccctggaagtatgtcatcaacacctgcgggcaagactttcccctgaaaaccaacagggaaatagttcagtatctgaagggatttaaagggaaaaatatcacccccggagtgctgcctcctgaccacgctgttggacggactaaatacgtccaccaagaactgttaaaccacaaaaattcctacgtgattaaaacaacaaaattaaaaactcctcctcctcatgacatggtgatttactttggcacggcctacgtggctctcacaagggactttgctaacttcgtcctccaagaccagctcgcacttgacttactctcctggtccaaggacacctacagccccgacgaacatttctgggtgacactcaacaggattcccggtgttcctggctctatgccaaatgcatcctggactggaaacctcagagctataaagtggagtgacatggaagacagacacggaggctgccacggccactatgtacatggtatttgtatctatggaaacggagacttaaagtggctggttaattcaccaagcctgtttgctaacaagtttgagcttaatacctacccccttactgtggaatgcctagaactgaggcatcgcgaaagaaccctcaatcagagtgaaactgcgatacaacccagctggtatttttgagctattcatgagctactcatgactgaagggaaactgcagctgggaagaggagcctgtttttgtgagagacttttgccttcgtaatgttaaccgtttcaggaccacgtttatagcttcaggacctggctacgtaattatacttaaaatatccactggacactgtgaaatacactaacaggatggctgggtagagcaatctgggcactttggccaattttagtcttgctgtttcttgatgctcacctctatattagtttattgttaggatcaatgataaatttaaatgacctcagatctttgcaccagatactcatcatatacaaatgttttagtaaaaaagagaattgtagataatactgtctaggaaaataagaattaggtttctttgaagaaggaatcttttataacaccttaacagtcaccactgtgctcaaccagacagatagtgaaacagctttctgggtaattcaccaatttcctttaaaacataagctacctgaatggagaatacatcttgtttctgagtttcaacactagcatttttggcttactcatggacaaagttctgtatatagtataaagtcattaacaagaaacaggatatgctttaagacagaattcactgtctgttgcttcagtaaaaggacctcggggaataaaacatttctctcttatatgccagaatgtaggctggtccctatgtcatgtcttccattaagaacactaaaaagtccttgcaagaatggagatatgcattcaagagaggtgctatcacatagatctagtctgaagtctggaacactttcctcttctatgacccctctctccccagtattatcttacttgcaaaatggagaccaaattctatcctgtgaggcttttaattgcaccatagtatgctctgagtagctttacactgcctggtactgatagtagtggctcgatttttaagagccttcaattgtagatgaacatctctgttatttatccctcattcatccatccgttcattcattcagccttcaatcaacatctcttgagtgtctattatgtacaggacatgtactgagacaaaaaggaaacataagagctttttcactctaaaaatcttggcaataatgtcaacaccagaaagcctcctctggagaatcttacagagtgattgtagtttaatacaggaacacacagggctgtgtagcatgataccaggcccaggagatcagtaattacaaattaagggttaaatcagagattattcaacagagagggagaaaggaggagacagagggaggacctgttgtgttccagccattctggtattcctttatgtatctaatttcattcaaacctcacaacagtcttgtgaggcccttatataattactcccattttgcagatgaagtaactgaggcttagaaaggttaatagcaccggggaacaatttctctgggtgagaattgggactctgttgctggtcttctcagttcatttcctgaggtggatttactgagagaaggtgaaataaagccatatttagtataccagagaaggtagattttaagaatggtctcagtgttaatactgagaaaaagtcctgtcagttcagaaaaaatgtgaagtctactttagtattcctgtaatactaaaccgttgagtttctaaatatttatttattctaacaaaaagcaattactacaaatggatgacacatttaatgaacacaattttattttttttctgtaactgtgcttgttgaatgtcaatcatatttaaagggaatgactttgaagtaaaaccttttttcttgctactgaaaaaaatggagttgttttgggtggtaaagtgttaaggaatagggacagctggtcacacaaggaactcttgaaggccacatgtgaaaacctgtcacttgcacagaggccagtcccactaaggtgaccagagtgggctccaagcacaaactgccattggctatagatgggactgtgtccccccaaaattcatgtgttggagccttaaccctcaatgtgatggtatttgagatggggcctttggtaagggaagtttagatgaggtcacgagggtaggaccctcatgatgggatgagtccccttacaagacctctggcttgggccgggcgtggtggctcacacctgtaatcccaacactttgggaggccaaggcaggtagatcacttgatgccaggagttccagaccaggctggccgacatggtgaaaccccatctctactaaaaaatataaaaattagccgggctttgtggcatgtgcctgtaatcccagctatttggcaggctgaggcatgagaatcgcttgaacccaggaggtggaggttacagtgagctgagagtgccccactgcactccagcctgggtgacagagcgagactttgtcccaaaacaaaataggtgaggggatagcgaatgcactcagggtcagcagtggagtttaaaaattgtctcttttcaacttatttaaatgacagcacctgagaagaggaaccgttttacactggatgtttctcatgtagaacaagaaatctttctggaattgatgtttacatgtctgttgttggtcatctctcctgtgtcttaaatactttaatgttggaagagcatagtgtttgggctagtgggtttctgacagcccatgggaatgccctgaaactactgtatctgatgtttgttttcgatgaggttccatgttttgttttcttgggaataaattaatatattgttttccaaaaaaaaaaaaaaaaaaaa

SEQ ID NO: 2 - protein, 402 aa, Homo sapiens
MMGSWKHCLFSASLISALIFVFVYNTELWENKRFLRAALSNASLLAEACHQIFEGKVFYPTENALKTTLDEATCYEYMVRSHYVTETLSEEEAGFPLAYTVTIHKDFGTFERLFRAIYMPQNVYCVHLDQKATDAFKGAVKQLLSCFPNAFLASKKESVVYGGISRLQADLNCLEDLVASEVPWKYVINTCGQDFPLKTNREIVQYLKGFKGKNITPGVLPPDHAVGRTKYVHQELLNHKNSYVIKTTKLKTPPPHDMVIYFGTAYVALTRDFANFVLQDQLALDLLSWSKDTYSPDEHFWVTLNRIPGVPGSMPNASWTGNLRAIKWSDMEDRHGGCHGHYVHGICIYGNGDLKWLVNSPSLFANKFELNTYPLTVECLELRHRERTLNQSETAIQPSWYF

(19) United States
(12) Patent Application Publication
(10) Pub. No.: US 2021/0130796 A1
(43) Pub. Date: May 6, 2021

(54) GCNT2/I-BRANCHING AS A BIOMARKER OF MELANOMA PROGRESSION

(71) Applicant: CHARLES J. DIMITROFF, CORAL GABLES, FL (US)

(72) Inventor: CHARLES J. DIMITROFF, CORAL GABLES, FL (US)

(73) Assignee: THE FLORIDA INTERNATIONAL UNIVERSITY BOARD OF TRUSTEES, MIAMI, FL (US)

(21) Appl. No.: 16/892,543

(22) Filed: Jun. 4, 2020

Related U.S. Application Data
(60) Provisional application No. 62/931,468, filed on Nov. 6, 2019.

(51) Int. Cl. C12N 9/10; A61P 35/00

(52) U.S. Cl. CPC C12N 9/1051; A61P 35/00; C12Y 204/0115

(57) ABSTRACT

The present invention provides methods and composition for diagnosis, prognosis, prevention and/or treatment of cancers such as melanomas. The subject invention provides biomarkers and methods for assessing the severity of a cancer/tumor and for monitoring the progressing of a cancer/tumor. The biomarkers include glycosylation-rerated genes and molecules affected by the glycosylation-rerated genes. The compositions according to the subject invention regulate malignancy-associated pathways and alter melanoma signaling, growth, and survival.

### CROSS-REFERENCE TO A RELATED APPLICATION

This application claims the benefit of U.S. provisional application Ser. No. 62/931,468, filed Nov. 6, 2019, which is incorporated herein by reference in its entirety.

### STATEMENT OF GOVERNMENT SUPPORT

This invention was made with government support under CA225644 awarded by the National Institutes of Health (NIH). The government has certain rights in the invention.

### SEQUENCE LISTING

The Sequence Listing for this application is labeled “SeqList-03Jun20-ST25.txt,” which was created on Jun. 3, 2020, and is 10 KB. The Sequence Listing is incorporated herein by reference in its entirety.

### BACKGROUND

Glycosylation is a common post-translational modification with more than 90% of cell-surface proteins and lipids being glycosylated. The glycome, or complete pattern of glycan modifications of a cell, is assembled by the sequential action of glycan-forming and glycan-degrading enzymes, glycosyltransferases and glycosidases, respectively, within the endoplasmic reticulum (ER) and Golgi apparatus. Compared with nucleotides and amino acids, glycans can be linked together in many different ways, thus glycans have vast structural complexity and heterogeneity. The numerous functions of glycans are based on their structural diversity and in most instances glycans “tune” function of a protein rather than turning it on or off. Glycans play an important role in maintain proper protein folding and extracellular matrix (ECM). Glycans are also key contributors in regulating intercellular and intracellular signaling, cell trafficking, host-pathogen interactions, and immune responses.

While a cancer cell's ability to proliferate, survive, generate a vascular bed, adapt to metabolic stress, evade the immune system, and metastasize are widely considered hallmarks of cancer, the dysregulated assembly and structure of glycans on cancer cells is still reluctantly acknowledged.

In cancer, altered cancer cell glycosylation can regulate numerous malignancy-associated pathways, including cell proliferation, death, migration/invasion, angiogenesis, metastasis, and immune evasion. Glycans represent the unifying “structural” thread through these functional activities, critical to the development and progression of cancer. By controlling cellular protein stability, membrane dynamics, subcellular trafficking, homo/heterophilic interactions, and extrinsic/intrinsic lectin-binding activities, cancer-associated glycans are uniquely poised to impact all virulent pathways.

Specifically, alterations in protein glycosylation are associated with malignant transformation and tumor progression. One of the most common tumor-associated glycan modifications is the truncation of serine/threonine O-linked glycans (T- and Tn-antigen). Specifically, truncated O-glycans directly induce oncogenic features leading to enhanced growth and invasion in pancreatic cancer, and poor outcomes in numerous other cancers. Besides truncated O-glycans, increased glycoprotein sialylation has also been shown to promote tumor growth, escape from apoptosis, resistance to therapy, and extravasation and seeding of circulating cancer cells through increased formation of sialyl Lewis X (sLex) glycans.

Moreover, increased size and complexity of asparagine (N-linked) glycans, predominantly via augmented expression or activity of N-acetylglucosaminyltransferase V (Mgat5), leads to protumorigenic galectin-ligand formation, enhanced cell motility and invasion, and increased metastatic potential in several cancers, including melanoma. Likewise, loss of N-linked glycosylation or presence of core fucosylation on certain signaling molecules, such as epidermal growth factor receptor (EGFR), neural cell adhesion molecule L1 (L1CAM), melanoma cell adhesion molecule (MCAM), vascular endothelial growth factor receptor 2 (VEGFR2) and integrins regulate receptor expression, dimerization, cleavage, lectin binding, and signaling in a variety of cancers.

Thus, although it is clear that aberrant glycans are present on cancer cells, the regulation of global glycosylation patterns in different cancers, and the functional/mechanistic ability of glycans to modulate tumor growth are largely unknown.

Therefore, there is a need to develop methods and compositions relating to glycosylation-related genes for diagnosis, prognosis, prevention and/or treatment of cancers, in particular, melanomas.

### BRIEF DESCRIPTION OF THE INVENTION

The present invention provides methods and composition relating to diagnosis, prognosis, prevention and/or treatment of cancers. The subject invention provides biomarkers and methods for assessing the severity of a cancer/tumor and for monitoring the progressing of a cancer/tumor. In a specific embodiment, the cancer is skin cancer such as melanoma.

In one embodiment, the subject invention identifies β-1,6 N-acetylglucosaminyltransferase 2 (GCNT2) and/or I-branched glycans that are involved in the pathogenesis of cancers. The methods according to the subject invention use GCNT2 and/or I-branched glycans as the biomarkers for diagnosing a cancer and/or assessing cancer progression.

Melanomas exhibit significant transcriptional changes in glycosylation-related genes. Compared with normal human epidermal melanocytes (NHEMs), GCNT2 is downregulated in metastatic melanomas. This leads to a loss of asparagine(N)-linked I-branched glycans and the synthesis of poly-N-acetyllactosamine (i-linear) glycans in melanomas.

Knockdown of GCNT2 significantly enhances melanoma xenograft growth and three-dimensional colony formation and survival, whereas increased expression of GCNT2 significantly decreases melanoma xenograft growth, and inhibits three-dimensional colony formation and survival. Also, GCNT2/I-branched glycan modifications inhibit insulin-like growth factor-1 (IGF-1) and ECM-mediated melanoma cell proliferation, survival, and associated downstream signaling pathways.

In one embodiment, the genes (e.g., GCNT2) and/or I-branched glycans of the present invention serve as biomarkers for: (1) the diagnosis of cancer; (2) the prognosis of cancer (e.g. monitoring cancer progression or regression from one biological state to another); (3) the susceptibility or prediction of response to treatment for a cancer; and/or (4) the evaluation of the efficacy to a treatment for a cancer.

In one embodiment, the subject invention provides methods for treating a cancer, e.g., melanoma, in a subject, comprising administering to the subject a composition comprising 1) a nucleic acid sequence that encodes GCNT2, 2) an amino acid sequence of GCNT2, 3) a vector comprising a nucleic acid sequence that encodes GCNT2, and/or 4) a cell that overexpresses a nucleic acid sequence of GCNT2 and/or an amino acid sequence of GCNT2.

In one embodiment, the present invention provides a method for the prevention of cancer, in particular, melanoma, comprising administering to a subject in need of such treatment a composition comprising 1) a nucleic acid sequence that encodes GCNT2, 2) an amino acid sequence of GCNT2, 3) a vector comprising a nucleic acid sequence that encodes GCNT2, and/or 4) a cell that overexpresses a nucleic acid sequence of GCNT2 and/or an amino acid sequence of GCNT2.

The subject invention also provide a pharmaceutical composition that comprises 1) a nucleic acid sequence that encodes GCNT2, 2) an amino acid sequence of GCNT2, 3) a vector comprising a nucleic acid sequence that encodes GCNT2, and/or 4) a cell that overexpresses a nucleic acid sequence of GCNT2 and/or an amino acid sequence of GCNT2.

In one embodiment, the present invention provides a method for assessing the progression of a cancer, e.g., melanoma, in a subject, the method comprising:

(i) assessing the expression level of GCNT2 and/or one or more I-branched glycans in a sample obtained from the subject;

(ii) comparing the expression level of GCNT2 and/or one or more I-branched glycans in the sample to a reference derived from the expression level of GCNT2 and/or one or more I-branched glycans in samples obtained from healthy subjects and determining the condition of the subject; and

(iii) for the subject determined to suffer from the cancer, e.g., melanoma, periodically repeating steps (i) and (ii) during treatment as a basis to determine the efficacy of said treatment by assessing whether the expression level of GCNT2 and/or one or more I-branched glycans in the subject is up-regulated or down-regulated, wherein an up-regulation in the expression level of GCNT2 and/or one or more I-branched glycans correlates to an improvement in the subject's condition.

In one embodiment, the present invention provides a method for assessing the progression of a cancer, e.g., melanoma, in a subject, the method comprising:

(i) assessing the expression level of one or more i-linear glycan in a sample obtained from the subject;

(ii) comparing the expression level of one or more i-linear glycan in the sample to a reference derived from the expression level of one or more i-linear glycan in samples obtained from healthy subjects and determining the current condition of the subject; and

(iii) for the subject determined to suffer from the cancer, e.g., melanoma, periodically repeating steps (i) and (ii) during treatment as a basis to determine the efficacy of said treatment by assessing whether the expression level of one or more I-branched glycans in the subject is up-regulated or down-regulated, wherein a down-regulation in the expression of one or more i-linear glycan correlates to an improvement in the subject's condition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-F show established cancer-associated glycans. The following cell surface carbohydrates on N- or O-glycans and their enzymatic regulators (in red) and respective nucleotide-sugar donor play key roles in cancer progression: (A) tri/tetra-antennary N-glycans (MGAT5), (B) truncated O-glycans, (C) bisecting GlcNAc N-glycans (MGAT3), (D)N-glycan core fucosylation (FUT8), (E) sialylated Lewis antigens, and (F) α2,6 sialylation (ST6Gal1).

FIGS. 2A-C show I-branched glycans and malignant progression. (A) I-branching activity of GCNT2 and subsequent β1,4 galactosyltransferase (β4GalT) activity on i-linear poly-LacNAc is depicted. The current models of GCNT2-regulated I-branched glycans driving the malignant or metastatic progression of breast, colon, and prostate cancer (B) or, alternatively, slowing the progression of malignant melanomas (C) are illustrated.

FIGS. 3A-B show GCNT2 staining intensity in patient death due to melanoma.

FIGS. 4A-B show GCNT2 expression in immunotherapy-resistant metastatic melanoma patients (non-responders) and immunotherapy-sensitive metastatic melanoma patients (responders).

### BRIEF DESCRIPTION OF SEQUENCES

SEQ ID NO: 1 is the nucleic acid sequence of GCNT2 contemplated for use according to the subject invention.

SEQ ID NO: 2 is the amino acid sequence of GCNT2 contemplated for use according to the subject invention.

### DETAILED DESCRIPTION OF THE INVENTION

The present invention provides methods and compositions for diagnosis, prognosis, prevention and/or treatment of cancers. The subject invention provides biomarkers and methods for assessing the severity of a cancer/tumor and for monitoring the progressing of a cancer/tumor. The biomarkers include glycosylation-related genes and molecules affected by the glycosylation-rerated genes. The subject invention also provides compositions for treating a cancer/tumor, for preventing or reducing the progression of a cancer/tumor. The subject invention further provide compositions for inhibiting the growth of primary melanomas, inhibiting metastasis, inhibiting the growth of metastases, killing circulating melanoma cells, inducing remission, extending remission, and/or inhibiting recurrence.

In one embodiment, the cancers exhibit significant transcriptional changes in glycosylation-related genes. In a specific embodiment, the cancer is a skin cancer such as melanoma. Melanoma is one of the most aggressive forms of cancer, typically beginning in the skin and often metastasizing to vital organs and other tissues. Melanomas include, but are not limited to, superficial spreading melanoma (SSM), nodular melanoma (NM), Lentigo maligna, lentigo maligna melanoma (LMM), mucosal melanoma, polypoid melanoma, desmoplastic melanoma, amelanotic melanoma, soft-tissue melanoma, uveal melanoma and acral lentiginous melanoma (ALM).

In one embodiment, melanoma may be a stage 0, I, II, III or IV melanoma. Stage 0 melanoma is a very early stage disease known as melanoma in situ. The tumor is limited to the epidermis with no invasion of surrounding tissues, lymph nodes, or distant sites. Stage 0 melanoma is considered to be very low risk for disease recurrence or spread to lymph nodes or distant sites.

Stage I melanoma is characterized by tumor thickness, presence and number of mitoses, and ulceration status. Stage I melanomas are considered to be low-risk for recurrence and metastasis. Sentinel lymph node biopsy is recommended for Stage I tumors thicker than 1.0 mm and for any ulcerated tumors of any thickness. Surgery is a common treatment for Stage I melanoma.

Stage II melanomas also are localized tumors characterized by tumor thickness and ulceration status. Stage II melanoma is considered to be intermediate-risk for local recurrence or distant metastasis. In addition to biopsy and surgery as described for Stage I, Stage II treatment may include adjuvant therapy, which is a treatment given in addition to a primary cancer treatment, following surgery. Treatments may include interferons therapies (e.g., interferon alfa-2a, and/or alfa-2b), and vaccines therapy.

Stage III melanomas are tumors that have spread to regional lymph nodes, or have developed in transit metastasis or satellites. Stage III disease is considered to be intermediate- to high-risk for local recurrence or distant metastasis. In addition to surgery and adjuvant therapy as described above, Stage III melanoma treatment often includes therapeutic lymph node dissection (TLND) to remove regional lymph nodes from the area where cancerous lymph nodes were found. The goal of the surgery is to prevent further spread of the disease through the lymphatic system.

Stage IV melanomas often are associated with metastasis beyond the regional lymph nodes to distant sites in the body. Common sites of metastasis are vital organs (lungs, abdominal organs, brain, and bone) and soft tissues (skin, subcutaneous tissues, and distant lymph nodes). Stage IV melanoma may be characterized by the location of the distant metastases; the number and size of tumors; and the serum lactate dehydrogenase (LDH) level. Elevated LDH levels usually indicate that the tumor has spread to internal organs. Treatments may include surgery to remove cancerous tumors or lymph nodes that have metastasized to other areas of the body, systemic therapies and radiation therapy.

Visual diagnosis of melanomas is still the most common method employed by health professionals. Metastatic melanomas can be detected by X-rays, CT scans, MRIs, PET and PET/CTs, ultrasound, LDH testing and photoacoustic detection.

In one embodiment, the subject invention identifies GCNT2 and/or I-branched glycans that are involved in the pathogenesis of melanomas. The methods according to the subject invention use DCNT2 and/or I-branched glycans as a biomarker for cancer diagnosis and/or progression. The subject invention also relates to the role of aberrant glycans in melanoma and changes in glycan structures that regulate different malignancy-associated pathways to alter melanoma cell growth and survival.

In one embodiment, the genes (e.g., GCNT2) and/or I-branched glycans of the present invention serve as biomarkers for: (1) the diagnosis of cancer; (2) the prognosis of cancer (e.g. monitoring cancer progression or regression from one biological state to another); (3) the susceptibility or prediction of response to treatment for a cancer; and/or (4) the evaluation of the efficacy to a treatment for a cancer.

For the diagnosis of a cancer, the level of the specific biomarker in a subject or a sample of the subject can be compared to a baseline or control level. If the level is below or above the control level, a certain cancer is implicated. The prognosis of a cancer can be assessed by comparing the level of the specific biomarker at a first time point to the level of the biomarker at a second time point that occurs at a given interval. The prediction of response to treatment for a cancer can be determined by obtaining the level of a specific biomarker and correlating this level to a standard curve. The evaluation of the efficacy of the treatment for a cancer can be assessed by comparing the level of the specific biomarker before administration of the treatment to the level of the biomarker after the administration of the treatment.

Expression of genes of the present invention can be measured by many methods known in the art. In general, expression of a nucleic acid molecule (e.g. RNA or DNA) can be detected by any suitable method or technique of measuring or detecting gene or polynucleotide sequence or expression. Such methods include, but are not limited to, polymerase chain reaction (PCR), reverse transcriptase PCR (RT-PCR), in situ PCR, quantitative PCR (q-PCR), in situ hybridization, flow cytometry, Western blot, Southern blot, Northern blot, immunohistochemistry, sequence analysis, microarray analysis, mass spectrometry analysis, detection of a reporter gene, or any other DNA/RNA hybridization platforms.

In one embodiment, the subject invention provides a method of identifying a cancer, e.g., melanoma, in a subject, the method comprising:

(a) determining the level of one or more biomarkers (e.g., GCNT2, I-branched glycans, MGAT3, MGAT5, B3GNT, and/or i-linear glycans) in:

i) a test sample obtained from the subject, and

ii) optionally, a control sample;

(b) optionally, obtaining at least one reference value corresponding to the level of one or more biomarkers (e.g., GCNT2, I-branched glycans, MGAT3, MGAT5, B3GNT, and/or i-linear glycans); and

(c) identifying the cancer, e.g., melanoma, in the subject based on the increased and/or reduced level of one or more biomarkers (e.g., GCNT2, I-branched glycans, MGAT3, MGAT5, B3GNT, and/or i-linear glycans) in the test sample and optionally, administering a therapy to the subject to treat and/or manage the cancer, e.g., melanoma.

The term “sample” as used herein refers to any physical sample that includes a cell or a cell extract from a cell, a tissue, a biofluid or an organ including a biopsy sample. The sample can be from a biological source such as a subject, or a portion thereof, or can be from a cell culture. Samples from a biological source can be from a normal or an abnormal organism, such as an organism known to be suffering from a condition or a disease state, or any portion thereof. Samples can also be from any fluid, tissue or organ including normal and abnormal (diseased) fluid, tissue or organ. Samples from a subject can be used, processed or cultured such that cells from the sample can be sustained in vitro as a primary or continuous cell culture or cell line.

In a specific embodiment, the sample is a skin sample, for example, skin cells, skin extract, and/or skin tissue. Preferably, the skin sample may comprise melanocytes.

The term “subject” or “patient,” as used herein, describes an organism, including mammals such as primates, to which diagnosis, prevention, assessment, and/or treatment according to the present invention can be provided. Mammalian species that can benefit from the disclosed methods of treatment include, but are not limited to, apes, chimpanzees, orangutans, humans, monkeys; domesticated animals such as dogs, cats; live-stocks such as horses, cattle, pigs, sheep, goats, and chickens; and other animals such as mice, rats, guinea pigs, and hamsters.

The terms “treatment” or any grammatical variation thereof (e.g., treat, treating, etc.), as used herein, includes but is not limited to, the application or administration to a subject (or application or administration to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical or mental well-being.

The term “prevention” or any grammatical variation thereof (e.g., prevent, preventing, etc.), as used herein, includes but is not limited to, at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease).

The term “prevention” may refer to avoiding, delaying, forestalling, or minimizing one or more unwanted features associated with a disease or disorder, and/or completely or almost completely preventing the development of a disease or disorder and its symptoms altogether. Prevention can further include, but does not require, absolute or complete prevention, meaning the disease or disorder may still develop at a later time and/or with a lesser severity than it would without preventative measures. Prevention can include reducing the severity of the onset of a disease or disorder, and/or inhibiting the progression thereof.

In one embodiment, the subject invention provides a detection method for determining the initiation of a systemic treatment for a cancer, e.g., melanoma, in a subject. The method comprises:

(a) determining the level of one or more biomarkers (e.g., GCNT2, I-branched glycans, MGAT3, MGAT5, B3GNT, and/or i-linear glycans) in:

i) a test sample obtained from the subject, and

ii) optionally, a control sample;

(b) optionally, obtaining at least one reference value corresponding to the level of one or more biomarkers (e.g., GCNT2, I-branched glycans, MGAT3, MGAT5, B3GNT, and/or i-linear glycans);

(c) determining whether or not to initiate the systemic treatment for the cancer, e.g., melanoma, in the subject based on a change in expression level of one or more biomarkers (e.g., GCNT2, I-branched glycans, MGAT3, MGAT5, B3GNT, and/or i-linear glycans) in the test sample; and

(d) administering a systemic treatment or therapy to the subject to treat and/or manage the cancer, e.g., melanoma.

In a further embodiment, the systemic treatment could be Immune Checkpoint Inhibitors (ICIs), e.g., anti-PD1, anti-PDLL and/or anti-CTLA4 treatments.

Immune checkpoints are known in the art and the term is well understood in the context of cancer therapy. Immune checkpoints include, but are not limited to, cytotoxic T-lymphocyte antigen 4 (CTLA-4), programmed cell death protein 1 (PD-1) and its ligand PDL-1, T-cell immunoglobulin and mucin domain-containing protein 3 (TIM-3), killer cell immunoglobulin-like receptor (KIR), lymphocyte activation gene-3 (LAG-3), V-domain immunoglobulin suppressor of T cell activation (VISTA), and B and T lymphocyte attenuator (BTLA). Inhibitors of immune checkpoints inhibit their normal immunosuppressive function, for example, by down regulating the expression of checkpoint molecules or by binding thereto and blocking normal receptor/ligand interactions. As a result, inhibitors of immune checkpoints enhance the immune response to an antigen, in particular, from a tumor cell.

Inhibitors of immune checkpoints are known in the art and preferred are anti-immune checkpoint antibodies, such as anti-CTLA-4 antibodies (e.g. ipilimumab and tremelimumab), anti-PD-1 antibodies (e.g. nivolumab, lambrolozumab, pidilizumab and RG7446 (Roche)) and anti-PDL-1 antibodies (e.g. BMS-936559 (Bristol-Myers Squibb), MPDL3280A (Genentech), MSB0010718C (EMD-Serono) and MED14736 (AstraZeneca)).

With knowledge of an immune checkpoint target, a skilled artisan is able to develop an inhibitor thereof. Inhibitors may be selected from proteins, peptides, peptidomimetics, peptoids, antibodies, antibody fragments, small inorganic molecules, small non-nucleic acid organic molecules or nucleic acids such as anti-sense nucleic acids, small interfering RNA (siRNA) molecules or oligonucleotides. The inhibitor may for example be a modified version of the natural ligand (e.g. for CTLA-4, CD80 (B7-1) and CD86 (B7-2)), such as a truncated version of one of the ligands. They may be naturally occurring, recombinant or synthetic.

In one embodiment, the subject invention provides methods for treating a cancer, e.g., melanoma, in a subject. The method comprises:

(i) assessing the expression level of one or more biomarkers selected from GCNT2, MGAT3, MGAT5, B3GNT, i-linear glycans and I-branched glycans in a sample obtained from the subject;

(ii) comparing the expression level of one or more biomarkers in the sample to a reference derived from the expression level of one or more biomarkers in samples obtained from healthy subjects;

(iii) identifying the cancer, e.g., melanoma, in the subject based on the increased level and/or reduced level of one or more biomarkers in the test sample; and

(iv) administering a systemic treatment to the subject, the systemic treatment comprising administering to the subject one or more immune checkpoint inhibitors (ICIs).

In one embodiment, the subject invention provides a method for treating a cancer, e.g., melanoma, in a subject, the method comprising:

(a) determining the level of one or more biomarkers (e.g., GCNT2, I-branched glycans, MGAT3, MGAT5, B3GNT, and/or i-linear glycans) in:

i) a test sample obtained from the subject, and

ii) optionally, a control sample;

(b) optionally, obtaining at least one reference value corresponding to the level of one or more biomarkers (e.g., GCNT2, I-branched glycans, MGAT3, MGAT5, B3GNT, and/or i-linear glycans);

(c) identifying the cancer, e.g., melanoma, in the subject based on the increased and/or reduced level of one or more biomarkers (e.g., GCNT2, I-branched glycans, MGAT3, MGAT5, B3GNT, and/or i-linear glycans) in the test sample; and

(d) administering a therapy to the subject to treat and/or manage the cancer, e.g., melanoma.

In one embodiment, the therapy to the subject to treat cancer, e.g., melanoma, may comprise administering to the subject a pharmaceutically effective amount of 1) a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 2) an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2, 3) a vector comprising a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 4) a cell that overexpresses a nucleic acid sequence of GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, and/or 5) a cell that overexpresses an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%; 97%, 98% or 99% identity with GCNT2.

In another embodiment, the therapy to the subject to treat cancer, e.g., melanoma, may further comprise administering to the subject a pharmaceutically effective amount of 1) a nucleic acid sequence that encodes B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT, 2) an amino acid sequence of B3GNT protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with B3GNT, 3) a vector comprising a nucleic acid sequence that encodes B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT, 4) a cell that overexpresses a nucleic acid sequence of B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT, and/or 5) a cell that overexpresses an amino acid sequence of B3GNT protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with B3GNT. In a specific embodiment, B3GNT comprises the human WT B3GNT.

A “nucleic acid” according to the invention refers to polynucleotides, such as DNA, RNA, modified DNA, modified RNA as well as mixtures thereof.

As used herein, “variants” of a protein refer to sequences that have one or more amino acid substitutions, deletions, additions, or insertions. In preferred embodiments, these substitutions, deletions, additions or insertions do not materially adversely affect the protein activity. Variants that retain one or more biological activities are within the scope of the present invention.

“Fragments” and its variants are also within the scope of proteins of the subject invention, so long as the fragment retains one or more biological properties. Preferably, the fragment is at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the full length protein, e.g., GCNT2 an B3GNT.

In one embodiment, the control sample is obtained from: i) an individual belonging to the same species as the subject and not having melanoma, or ii) the subject at a prior time known to be free from melanoma.

In one embodiment, the nucleic acid sequence of GCNT2 comprises, or consists of, a sequence of Accession No. NM_145649 (SEQ ID NO: 1), or a nucleic acid sequence sharing at least 50%, 55%, 60%, 65%, 70%, 75%, 80%, 85%, 90%, 95%, 96%, 97%, 98% or 99% identity with Accession No. NM_145649.

In one embodiment, the amino acid sequence of GCNT2 comprises, or consists of, a sequence of Accession No. NP_663624 (SEQ ID NO: 2) or an amino acid sequence sharing at least 50%, 55%, 60%, 65%, 70%, 75%, 80%, 85%, 90%, 95%, 96%, 97%, 98% or 99% identity with Accession No. NM_663624.

The subject invention provide a pharmaceutical composition comprises 1) a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 2) an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2, 3) a vector comprising a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 4) a cell that overexpresses a nucleic acid sequence of GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, and/or 5) a cell that overexpresses an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2.

In one embodiment, the pharmaceutical composition further comprises 1) a nucleic acid sequence that encodes B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT, 2) an amino acid sequence of B3GNT protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with B3GNT, 3) a vector comprising a nucleic acid sequence that encodes B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT, 4) a cell that overexpresses a nucleic acid sequence of B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT, and/or 5) a cell that overexpresses an amino acid sequence of B3GNT protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with B3GNT.

In one embodiment, the composition according to the subject invention also comprises a pharmaceutically acceptable carrier. “Pharmaceutically acceptable carrier” refers to a diluent, adjuvant or excipient with which the antigen disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith. Examples of carriers suitable for use in the pharmaceutical compositions are known in the art and such embodiments are within the purview of the invention.

The compositions of the present invention can be administered to the subject being treated by standard routes, including the local, oral, ophthalmic, nasal, topical, intratumoural, transdermal, intra-articular, parenteral (e.g., intravenous, intraperitoneal, intradermal, subcutaneous or intramuscular), intracranial, intracerebral, intraspinal, intravaginal, intrauterine, or rectal route. Additionally, the composition may be administered directly into the tumor of melanoma. Depending on the condition being treated, one route may be preferred over others, which can be determined by those skilled in the art.

In one embodiment, the composition is formulated in accordance with routine procedures as a pharmaceutical composition adapted for local administration to human beings. Typically, compositions for local administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile water for injection or saline can be provided so that the ingredients may be mixed prior to administration.

The present invention provides novel and advantageous therapeutic methods for treating cancer, in particular, melanoma, comprising administering to a subject in need of such treatment a pharmaceutical composition comprising 1) a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 2) an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2, 3) a vector comprising a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 4) a cell that overexpresses a nucleic acid sequence of GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, and/or 5) a cell that overexpresses an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2.

In one embodiment, the methods for treating cancer, in particular, melanoma, comprises administering to a subject in need of such treatment a pharmaceutical composition comprising:

1) a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, and/or a nucleic acid sequence that encodes B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT;

2) an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2, and/or an amino acid sequence of B3GNT protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with B3GNT;

3) a vector comprising a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, and/or a nucleic acid sequence that encodes B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT;

4) a cell that co-overexpresses a nucleic acid sequence of GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, and/or a nucleic acid sequence of B3GNT or a nucleic acid sequence sharing at least 50%, 55%, 60%, 65%, 70%, 75%, 80%, 85%, 90%, 95%, 96%, 97%, 98% or 99% identity with the nucleic acid sequence that encodes B3GNT; and/or

5) a cell that co-overexpresses an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2, and/or an amino acid sequence of B3GNT protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with B3GNT.

In one embodiment, the method of treating/preventing/reducing the progression of melanoma may further comprise administering to the subject one or more therapeutic agents. The therapeutic agent may comprise a chemotherapeutic agent, immunotherapeutic agent, gene therapy or radio therapeutic agent.

In one embodiment, the pharmaceutical composition of the subject invention may further comprise one or more therapeutic agents. The therapeutic agent may comprise a chemotherapeutic agent (e.g., dacarbazine or cisplatin), immunotherapeutic agent (e.g., interleukin-2 (IL-2) or interferon (IFN)), gene therapy and/or radio therapeutic agent. The therapeutic agent may further comprise other cytotoxic agents such as anti-tumour peptides, cytokines e.g. IFN-γ, TNF, CSF and growth factors, and/or cancer vaccines.

In one embodiment, dosage units containing the nucleic acid and/or peptidic molecules contain about 0.01 mg to 1000 mg, about 0.01 mg to 900 mg, about 0.01 mg to 800 mg, about 0.01 mg to 700 mg, about 0.01 mg to 600 mg, about 0.01 mg to 500 mg, about 0.05 mg to 500 mg, about 0.1 mg to 400 mg, about 0.1 mg to 300 mg, about 0.1 mg to 200 mg, about 0.1 mg to 100 mg, about 0.1 mg to 90 mg, about 0.1 mg to 80 mg, about 0.1 mg to 70 mg, about 0.1 mg to 60 mg, about 0.1 mg to 50 mg, about 0.1 mg to 40 mg, about 0.1 mg to 30 mg, about 0.1 mg to 20 mg, about 0.1 mg to 10 mg, about 0.5 mg to 50 mg, about 1 mg to 40 mg, about 1 mg to 20 mg, about 1 mg to 10 mg, or about 1 mg to 5 mg.

In one embodiment, the composition may be formulated for administration as tablets, coated tablets, nasal sprays, solutions, emulsions, liposomes, powders, capsules or sustained release forms.

In specific embodiments, the composition of the subject invention may be administered at least once a day, twice a day, or three times a day for consecutive days, e.g. 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. The composition of the subject invention may also be administered for weeks, months or years.

In one embodiment, the present invention provides a method for the prevention of cancer, in particular, melanoma, comprising administering to a subject in need of such treatment a pharmaceutical composition according the subject invention.

In one embodiment, the methods according to the subject invention may further comprise a step of determining the levels of one or more biomarkers (e.g., GCNT2, I-branched glycans, MGAT3, MGAT5, B3GNT, and/or i-linear glycans) in a sample of the subject prior to the administration and/or after the administration.

In one embodiment, the present invention provides a method for treating a subject having a risk of developing melanoma, the method comprising administering to a subject in need of such treatment a pharmaceutical composition according the subject invention.

A further embodiment of the invention provides a method for monitoring the effect of a treatment for a cancer, such as melanoma, in a subject. A method for monitoring the effect of a treatment for a cancer, such as melanoma in a subject may comprise:

(a) determining the level of one or more biomarkers (e.g., GCNT2, I-branched glycans, MGAT3, MGAT5, B3GNT, and/or i-linear glycans) in:

i) a pre-treatment test sample obtained from the subject before the treatment,

ii) a post-treatment test sample obtained from the subject after the treatment, and

ii) optionally, a control sample;

(b) optionally obtaining at least one reference values corresponding to levels of one or more biomarkers (e.g., GCNT2, I-branched glycans, MGAT3, MGAT5, B3GNT, and/or i-linear glycans); and

(c) identifying the treatment for the cancer, e.g., melanoma, in the subject as effective based on the levels of one or more biomarkers (e.g., GCNT2, I-branched glycans, MGAT3, MGAT5, B3GNT, and/or i-linear glycans) in the post-treatment test sample compared to the levels of one or more biomarkers (e.g., GCNT2, I-branched glycans, MGAT3, MGAT5, B3GNT, and/or i-linear glycans) in the pre-treatment test sample and optionally, continuing the treatment for the chronic pulmonary disease in the subject, or

(d) identifying the treatment for the cancer, e.g., melanoma in the subject as ineffective based on the levels of one or more biomarkers (e.g., GCNT2, I-branched glycans, MGAT3, MGAT5, B3GNT, and/or i-linear glycans) in the post-treatment test sample compared to the levels of one or more biomarkers (e.g., GCNT2, I-branched glycans, MGAT3, MGAT5, B3GNT, and/or i-linear glycans) in the pre-treatment test sample and optionally, modifying the treatment in the subject.

In a preferred embodiment, the melanoma is an ICI therapy-resistant melanoma.

In one embodiment, the subject invention provides a method for diagnosing and/or assessing the progression of melanoma in a subject, the method comprising:

(i) assessing the expression level of one or more biomarkers in a sample obtained from the subject;

(ii) comparing the expression level of one or more biomarkers in the sample to a reference derived from the expression level of one or more biomarkers in samples obtained from healthy subjects; and

(iii) determining the progression of melanoma in the subject based on whether the expression level of one or more biomarkers in the subject is up-regulated or down-regulated;

wherein the biomarkers may be selected from, for example, GCNT2, i-linear glycans, MGAT3, MGAT5, B3GNT, I-branched glycans and any combination thereof.

In one embodiment, the present invention provides a method for diagnosing and/or assessing the progression of a cancer, e.g., melanoma, in a subject who may be undergoing treatment for the cancer, e.g., melanoma, the method comprising:

(i) assessing the expression level of one or more biomarkers in a sample obtained from the subject;

(ii) comparing the expression level of one or more biomarkers in the sample to a reference derived from the expression level of one or more biomarkers in samples obtained from healthy subjects and determining the current condition of the subject; and

(iii) for the subject determined to suffer from the cancer, e.g., melanoma periodically repeating steps (i) and (ii) during treatment as a basis to determine the efficacy of said treatment by assessing whether the expression level of one or more biomarkers in the subject is up-regulated or down-regulated, wherein the biomarkers are selected from, GCNT2, i-linear glycans, MGAT3, MGAT5, B3GNT, I-branched glycans and any combination thereof.

In further embodiments, the biomarker is GCNT2 and an up-regulation in the expression level of GCNT2 in the sample is indicative of an improvement in the subject's condition; the biomarker is I-branched glycan and an up-regulation in the expression level of I-branched glycan in the sample is indicative of an improvement in the subject's condition; the biomarker is i-linear glycan and a down-regulation in the expression level of i-linear glycan in the sample is indicative of an improvement in the subject's condition; and/or the biomarker is B3GNT and a down-regulation in the expression level of B3GNT in the sample is indicative of an improvement in the subject's condition.

Compared to NHEMs, melanomas downregulate the glycosyltransferase, GCNT2, and display a corresponding loss of I-branched glycans on the cell surface. The N-glycome of NHEMs contains high mannose, N-acetyllactosamines (LacNAcs) appearing as chains of repeating units of N-acetylglucosamine and galactose (polyLacNAcs), and complex N-glycan structures. NHEM polyLacNAcs are further modified with branched LacNAcs, known as I-branches. In contrast, melanoma cells, for example, A375 and G361 cell contain polyLacNAcs that are typically displayed in long linear chains (i-linear glycans). The downregulation of GCNT2 in melanoma cells is particularly important because GCNT2 is the enzyme that catalyzes the transfer of N-acetylglucosamine to galactose residues on polyLacNAcs to form I-branched glycans.

In clinical specimens, GCNT2 expression inversely correlated with melanoma progression. GCNT2 gene expression levels also directly correlated with the presence of cell surface I-branched glycans. Loss of GCNT2/I-branched glycans promoted melanoma xenograft growth, colony formation, and cell survival, while overexpression of GCNT2/I-branched glycans negatively regulated melanoma xenograft growth, colony formation, and cell survival.

In one embodiment, low GCNT2/I-branched glycan expression increased melanoma cell proliferation and survival by enhancing IGF1R- and integrin:ECM-mediated signaling. GCNT2/I-branched glycans decreased IGF-1 and RGD ligand binding activity on melanoma cells, suggesting that GCNT2/I-branches may modulate IGF1R and fibronectin: integrin signaling through modulation of ligand binding capacity. Loss of GCNT2/I-branched glycans in melanomas regulates multiple cell surface glycoprotein signaling pathways and promotes melanoma growth and survival.

In specific embodiments, the I-branched glycans are N-glycans comprising LacNAc. The I-branched glycan comprising Galß1,4GlcNAc moieties. The i-linear glycans are N-glycans comprising linear polyLacNAc. Also, the i-linear glycans may be N-glycans comprising (Galβ1,4-GlcNAcβ1,3)n (n≥2).

Moreover, melanomas express longer polyLacNAcs than normal melanocytes. The synthesis of longer polyLacNAcs is also important contributor to malignancy. Thus, the expression level of β1,3-glucosaminyltransferase (B3GNT) genes, which encode enzymes that extend polyLacNAcs, may increase in melanomas compared with normal melanocytes.

In one embodiment, the method of the subject invention may further comprise determining the level of one or more B3GNT genes in:

i) a test sample obtained from the subject, and

ii) optionally, a control sample;

optionally, obtaining at least one reference value corresponding to the level of one or more B3GNT genes; and

identifying the cancer, e.g., melanoma, in the subject based on the increased level of one or more B3GNT genes in the test sample and optionally, administering a therapy to the subject to treat and/or manage the cancer, e.g., melanoma, wherein the therapy comprises administering one or more inhibitors of one or more B3GNT.

Furthermore, extended polyLacNAcs could potentially be regulated by B3GNT and GCNT2 competition for the same nucleotide donor sugar, UDP-GlcNAc, thereby limiting polyLacNAc length when GCNT2 is expressed. Normal melanocytes can have extended polyLacNAcs, consisting of four or more LacNAc residues, which are modified with I-branched glycans. The downregulation of GCNT2 is important for the loss of I-branched glycans in melanoma cells and perhaps even for the increase in extended i-linear polyLacNAcs through less donor sugar competition between B3GNT and GCNT2.

An intriguing possibility for the downregulation of GCNT2 and I-branched glycans in melanomas compared to NHEMs is that melanomas acquire these changes as part of a broader reversion to a more embryonic-like phenotype. In fact, erythrocytes, epithelial cells and dividing cells of the fetus predominately express i-linear glycans, which are thought to promote cell adhesion and proliferation during development, whereas in adults, i-linear glycans on these cells are largely replaced by I-branched glycans. Hence, the decrease in GCNT2 and increase in i-linear glycan expression on melanoma cells reflect a more dedifferentiated state compared to their normal counterpart.

The negative association of GCNT2 with metastasis suggests that loss of GCNT2/I-branched glycans may help melanomas progress. The majority of cancer deaths are attributed to the metastatic spread of cancer cells to visceral organs rather than to the primary tumor growth. Tumor cells remodel their cell-surface glycans to aid in the metastatic process by promoting dynamic interactions with ECM, migration through the circulation and lodgment/entry into distant tissues.

Tumor cell i-linear/I-branched glycan modifications as regulators of metastatic potential by controlling prosurvival and cell signaling activities. As such, detection of GCNT2/I-branched glycans as a biomarker can predict which patients are at risk for progression to metastatic disease.

In one embodiment, the subject invention provides a method of predicting a risk of melanoma progressing to a metastatic state, in a subject, the method comprising:

(a) determining the level of one or more biomarkers (e.g., GCNT2, I-branched glycans, MGAT3, MGAT5, B3GNT, and/or i-linear glycans) in:

i) a test sample obtained from the subject, and

ii) optionally, a control sample;

(b) optionally, obtaining at least one reference value corresponding to the level of one or more biomarkers (e.g., GCNT2, I-branched glycans, MGAT3, MGAT5, B3GNT, and/or i-linear glycans); and

(c) determining the risk of melanoma progressing to a metastatic state in the subject based on the level of one or more biomarkers (e.g., GCNT2, I-branched glycans, MGAT3, MGAT5, B3GNT, and/or i-linear glycans) in the test sample and optionally, administering a therapy to the subject to treat and/or manage the progression.

In one embodiment, the present invention provides a method for accessing the progression of a cancer, e.g., melanoma, in a subject who may or may be undergoing treatment for the cancer, e.g., melanoma, the method comprising:

(i) assessing the expression level of GCNT2 and/or i-linear glycan in a sample obtained from the subject;

(ii) comparing the expression level of GCNT2 and/or i-linear glycan in the sample to a reference derived from the expression level of GCNT2 and/or i-linear glycan in samples obtained from healthy subjects and determining the current condition of the subject; and

(iii) for the subject determined to suffer from the cancer, e.g., melanoma periodically repeating steps (i) and (ii) during treatment as a basis to determine the efficacy of said treatment by assessing whether the expression level of GCNT2 and/or I-branched glycans in the subject is up-regulated or down-regulated, wherein an up-regulation in the expression level of GCNT2 and/or a down-regulation in the expression of i-linear glycan correlates to an improvement in the subject's condition.

PolyLacNAcs often serve as ligands for β-galactoside-binding lectins, known as galectins, and GCNT2 modifies polyLacNAcs. GCNT2/I-branched glycan expression decreased IGF-1 and RGD ligand binding activities on the surface of melanoma cells. There are several reasons for how GCNT2/I-branched glycans regulate downstream signaling, including but not limited to, cell surface receptor expression level, receptor-ligand binding, and membrane organization, including dimerization and clustering. GCNT2/I-branched glycans displayed by N-glycans at key glycosylation sites on particular glycoproteins, such as IGF1R or integrins, may influence ligand induced conformational changes required for receptor signal transduction. Alternatively, as many cell surface receptors, including IGF1R and integrins, undergo receptor dimerization or clustering upon ligand binding, presence of I-branched glycans, could inhibit the ability of these receptors to complex efficiently.

In one embodiment, the subject invention also provides a method for assessing the chance of survival/death of a melanoma patient, the method comprising:

(a) determining the level of one or more biomarkers in:

i) a test sample obtained from the subject, and

ii) optionally, a control sample;

(b) optionally, obtaining at least one reference value corresponding to the level of one or more biomarkers; and

(c) determining the chance of survival/death of the melanoma patient based on the change in the level of one or more biomarkers in the test sample; and

(d) optionally, administering a therapy to the subject to treat and/or manage melanoma, wherein the biomarkers are selected from GCNT2, i-linear glycans, MGAT3, MGAT5, B3GNT, I-branched glycans and any combination thereof.

In a specific embodiment, the melanoma patient is at least stage 2, 3, or 4.

In some embodiments, glycans may be detect from methods, for example, using a dearth of anticarbohydrate antibodies or plant lectins, and/or using matrix-assisted laser/desorption ionization time-of-flight mass spectrometry.

In one embodiment, the subject invention provides a method for increasing I-branched glycans in a melanoma cell, the method comprising contacting the melanoma cell with a composition according to the subject invention. In a further embodiment, the method comprises contacting 1) a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2; 2) an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2; and/or 3) a vector comprising a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2. Such method results in an overexpression of GCNT2 in the melanoma cells.

In one embodiment, the subject in need of the treatment for melanoma, preferably, ICI therapy-resistant melanoma, has been treated by ICI or IC therapy.

In one embodiment, the subject invention provides a method for treating an IC therapy-resistant melanoma in a subject, the method comprising administering to the subject a pharmaceutical composition of the subject invention. Preferably, the composition comprising 1) a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 2) an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2, and/or 3) a vector comprising a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2.

In one embodiment, the subject invention further provides a method for increasing/enhancing/improving the sensitivity of a subject having melanoma to an IC therapy, the method comprising administering to the subject a pharmaceutical composition of the subject invention.

In a further embodiment, the pharmaceutical composition may be administered prior to the administration of the IC therapy, simultaneously with the IC therapy, or after the administration of the IC therapy.

In a preferred embodiment, the IC therapy is an anti-PD-1 therapy.

In one embodiment, the subject invention provides a method for increasing/enhancing/improving the sensitivity of melanoma cells to an ICI, the method comprising contacting the melanoma cells with a pharmaceutical composition of the subject invention.

Contacting the melanoma cells with such composition results in, for example, the transfection or transduction of the GCNT2 gene into the melanoma cells, which leads to overexpression of GCNT2 in these cells. There are various transfection methods, including physical treatment (e.g., electroporation microinjection, cell squeezing, impalefection, hydrostatic pressure, continuous infusion, sonication, nanoparticles, and magnetofection), chemical materials (e.g., lipofection, and polyplexes) or biological particles (e.g., retrovirus, lentivirus, adenovirus, adeno-associated virus, and herpes simplex virus) that are used as carriers.

In one embodiment, the ICI therapy-resistant melanoma or melanoma cells are not responsive to the treatment of an ICI. Increasing the sensitivity of melanoma or melanoma cells to an ICI refers to, for example, reversing the resistance by at least 5%, 10%, 15%, 20%, 25%, 30%, 35%, or 40% so that the melanoma or melanoma cells are responsive to the treatment of the ICIs.

In one embodiment, the subject invention provides a method for slowing the malignant transformation, growth and/or metastasis of melanoma and/or melanoma cells, the method comprising contacting the melanoma/melanoma cells with a composition comprising 1) a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2, 2) an amino acid sequence of GCNT2 protein, biologically-active fragments, variants thereof, or an amino acid sequence sharing at least 50%, 55%, 60%, 65%, 70%, 75%, 80%, 85%, 90%, 95%, 96%, 97%, 98% or 99% identity with GCNT2, and/or 3) a vector comprising a nucleic acid sequence that encodes GCNT2 or a nucleic acid sequence sharing at least 50%, 55%, 60%, 65%, 70%, 75%, 80%, 85%, 90%, 95%, 96%, 97%, 98% or 99% identity with the nucleic acid sequence that encodes GCNT2.

In a further embodiment, the melanoma cells are resistant to one or more ICIs selected from, for example, antibodies to cytotoxic T-lymphocyte antigen 4 (CTLA-4), programmed cell death protein 1 (PD-1), PD-1 ligand (PDL-1), T-cell immunoglobulin and mucin domain-containing protein 3 (TIM-3), killer cell immunoglobulin-like receptor (KIR), lymphocyte activation gene-3 (LAG-3), V-domain immunoglobulin suppressor of T cell activation (VISTA), and B and T lymphocyte attenuator (BTLA).

In one embodiment, the subject invention provides a method for predicting an outcome of an IC therapy to a cancer, e.g., melanoma, in a subject, the method comprising:

(i) assessing the expression level of GCNT2 and/or i-linear glycan in a sample (e.g., tumor specimen) obtained from the subject;

(ii) comparing the expression level of GCNT2 and/or i-linear glycan in the sample to a reference derived from the expression level of GCNT2 and/or i-linear glycan in samples obtained from healthy subjects; and

(iii) determining/predicting the outcome of the IC therapy by assessing whether the expression level of GCNT2 and/or i-branched glycans in the subject is up-regulated or down-regulated, wherein an up-regulation in the expression level of GCNT2 and/or a down-regulation in the expression of i-linear glycan indicates that the cancer, e.g., melanoma, is responsive and/or sensitive to IC therapy.

### Glycans

Glycans on cancer cell include, but not limited to, bulky tri/tetraantennary N-glycans, truncated serine/threonine (O)-linked glycans, bisecting N-acetylglucosamine (GlcNAc)N-glycans, N-glycan core fucosylation, sialylated Lewis antigens, and α2,6 sialylation. These cancer-associated glycans can subtly tune a cancer cell's ability to proliferate, survive, invade, evade the immune system, and form distant metastases. Expression of cancer-associated glycans is conspicuously contingent on a distinct cell type with lineage-specific gene-expression patterns uniquely leveraged upon cellular transformation and malignant progression.

### Tri/Tetra-Antennary N-Glycans.

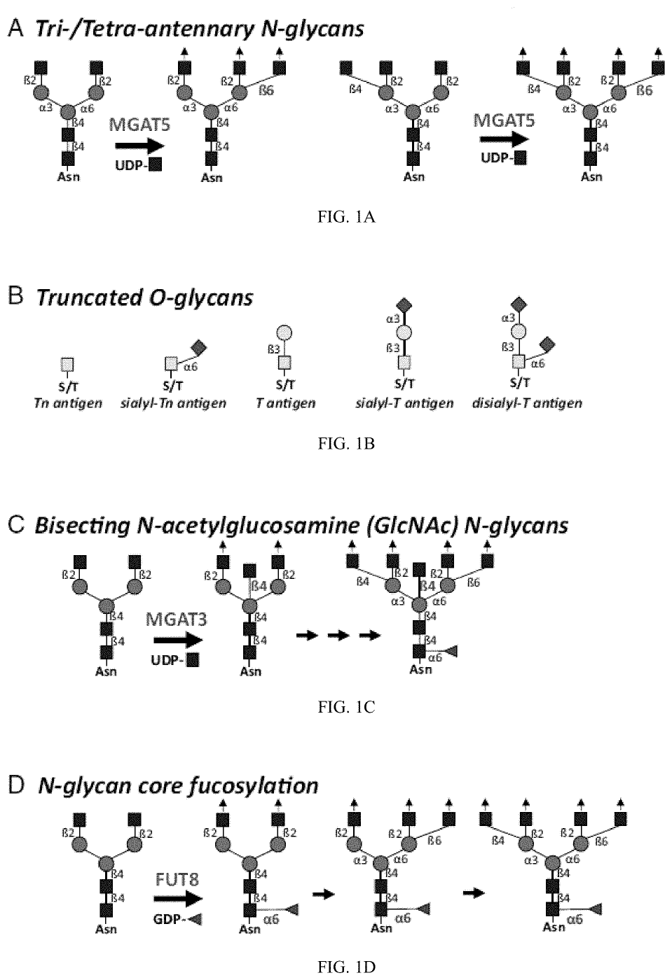

One of the most impactful posttranslational modifications on Golgi-derived membrane and secreted proteins is N-glycosylation. Notably, the enzymatic activity of α-mannosyl-β1,6 N-acetylglucosaminyltransferase-V (GnT-V; MGAT5) generates a bulky tri/tetra-antennary N-glycan species that can modify a protein's half-life, stability, membrane dynamics extracellular-binding partners, and functional activity (FIG. 1A). Elevations in MGAT5 and resultant large tri/tetra-antennary N-glycans can affect cancer cell virulence: MGAT5 expression promotes homo-/heterotypic adhesion and migratory activity, tumorigenicity, and metastasis in mouse models of breast and lung cancer. Specific MGAT5 N-glycan-dependent alterations on gastric cancer cells cause destabilization and aberrant membrane localization of Ecadherin and of adherens-junctions that impair homotypic cell-cell aggregation. MGAT5 overexpression in fibrosarcoma cells compromises N-cadherin clustering and signaling activity and increases cell motility via phosphorylation of catenins and reduces α5β1 clustering to enhance migration and invasion. Elevations in MGAT5 and tetra-antennary N-glycan levels correspond better with the fibronectin integrin receptor-mediated adhesion and motility of a metastatic melanoma cell line compared with the matching localized melanoma cell line variant. MGAT5-modified N-glycans often contain N-acetyllactosamine (LacNAc) moieties that bind galectins, form organized lattices, and accentuate promalignant activity of growth factor receptor tyrosine kinases (RTK) and integrins.

### Truncated O-Glycans.

O-glycosylations, another major Golgi-derived protein glycosylation modification, are represented by a series of 8 diverse core structures. O-glycan biosynthesis is initiated by addition of N-acetylgalactosamine (GalNAc) by one of 20 polypeptide N-acetylgalactosaminyltransferase family members to form a simple Tn antigen moiety. In cancer, enzymatic extension of Tn antigen with N-acetylneuraminic acid (NeuAc) or galactose (Gal) to generate sialo-Tn or core 1 O-glycans (T antigen) (FIG. 1b) or with β1,6 GlcNAc to build core 2 O-glycans that is often dysregulated and associated with numerous malignancies. The action of core 1 β1,3 galactosyltransferase 1 (C1GalT1) with the core 1 synthase chaperone Cosmic; α-GalNAc-α2,6 sialyltransferases-1, -2, -3 and -4 (ST6GalNAc1-4); α2,3 sialyltransferase 1 (ST3Gal-1); or core 2 β1,6 N-acetylglucosaminyltransferases 1 and 2 (GCNT1 or 3) are synthetically positioned to compete for these budding Tn and core 1 O-glycan acceptors, which are often aberrantly expressed and commonly related to cancer progression and poor prognosis. These enzymes function sequentially and often in competition for the same glycan acceptor to produce structurally diverse O-glycan species. For example, elevations in ST6GalNAc enzymes or expression of mutant nonfunctional Cosmic increase levels of sialo-Tn, whereas reductions in ST6GalNAc enzymes heighten core 2 O-glycan levels. Whether overexpressed or down-regulated depending on the cancer subtype, these O-glycan-modifying enzymes can function as critical biosynthetic regulators of siglec- or galectin-binding O-glycosylations. Cancer cells harness their dysregulated glycoenzyme signatures to preferentially yield truncated O-glycans, sialo-Tn, or sialo-core 1 or extended core 2 O-glycans, translating to siglec- or galectin-dependent malignant behaviors, respectively.

Cancer-associated truncated O-glycans have been directly linked with breast, ovarian, gastric, colorectal, and pancreatic malignancies and have been shown to impact several oncogenic features, including cell adhesion, migration and invasion, and immunoregulation. Furthermore, cancer-associated truncated O-glycans (or lack thereof) are also integral in modifying the binding activities of galectin (Gal)-1 and Gal-3 and of tumor-associated macrophage siglec-15 that, upon binding, render an intrinsic malignant activity or a TGF-β-dependent protumor immune microenvironment, respectively. That is, reductions in truncating O-glycan-modifying ST6GalNAc1-4 can elevate Gal-1 binding extended poly-LacNAc core 2 O-glycans, while elevations in these enzymes can increase Gal-3 binding core 1 O-glycans to help confer growth, adhesive and metastatic seeding activities.

### Bisecting GlcNAc N-Glycans.

Hybrid or biantennary complex Nglycans can be bisected with GlcNAc by β-mannosyl-β1,4 Nacetylglucosaminyltransferase-III (GnT-III; MGAT3) (FIG. 1c). While this GlcNAc addition is not typically elongated, it can theoretically impart molecular rigidity or a “spacer” moiety that affects how N-glycosylation impacts a protein's function. So, depending on cancer cell type, this N-glycan maturation step can either compromise or promote malignant activities. Lung metastatic activity of murine melanomas is lowered by MGAT3 overexpression; cancer cell growth factor receptor signaling is attenuated; and absence of MGAT3 in murine mammary tumors increases tumor growth, migration, and metastasis, whereas overexpression of MGAT3 inhibits early mammary tumor development and tumor cell migration. Bisecting GlcNAcs have also been shown to alter cancer cell E-cadherin and integrin receptor stability and function and boost Notch receptor activity related to ovarian cancer progression.

### N-Glycan Core Fucosylation.

Cell surface α1,3/4 fucosylation is best known for generating sialylated Lewis antigens, critical for cancer cell binding to endothelial (E)-selectin, vascular adhesion, and seeding in distant tissues. However, more recent data suggest that α1,6 fucosylation of the most proximal GlcNAc in the N-glycan chitobiose core by α1,6 fucosyltransferase 8 (FUT8) (FIG. 1d) is a key structure regulating the function of cancer cell membrane receptors. When FUT8 gene expression and resultant α1,6 fucosyl moieties are elevated, breast cancer cells exhibit an enhanced ability to signal through TGF-β receptor pathway and undergo malignancy-associated epithelial to mesenchymal transition and related metastatic activities. Similarly, core N-glycan α1,6 fucosylation on lung cancer cells enhances EGFR-dependent signaling activity and regulates E-cadherin-dependent nuclear translocation of β-catenin and, when silenced on melanoma cell adhesion molecules, suppresses invasion and tumor dissemination.

### Sialylated Lewis Antigens.

Sialylated Lewis antigens, α2,3 sialyl Lewis A (sLe^{A}) and α2,3 sialyl Lewis X (sLe^{X}), are elevated on aggressive cancer cells and linked to metastatic potential (FIG. 1e). The function of sLe^{X/A} on cancer cells is its ability to bind vascular endothelial (E)- and platelet (P)-selectins and promote vascular endothelial cell adhesion to help deliver circulating cancer cells to distant tissues. Cancer cell-selectin binding interactions characteristically yield tethering and rolling events on the luminal aspect of postcapillary venules that precede firm adherence and tissue entry, analogous to the leukocyte homing paradigm. While most cancer cells are enzymatically equipped to generate terminal α2,3 sialyl LacNAc moieties by ST3Gal3, ST3Gal4, and ST3Gal6 at the termini of their N-glycans, core 2 O-glycans, and neolacto glycosphingolipids, selectin-binding proficiency is consummated by the action of α1,3/4 fucosyltransferases (FUT3-7, 9-11) to synthesize sLe^{X} or sLe^{A} antigens. Whereas FUT3 and, to a minor extent, FUT5 exhibit α1,4 fucosyltransferase activity for sLe^{A} synthesis, FUT3, FUT5-7, and FUT9 predominantly provide the α1,3 fucosyltransferase activity necessary for synthesizing sLe^{X} and related selectin-binding activities. Uniformly, decades of experimental and correlative analyses indicate that a high level of sLe^{X} and sLe^{A} antigens inversely correlates with the survival of patients with most if not all types of malignancies. Cancer of the colon, breast, prostate, multiple myeloma, and pancreas commonly leverage their elevated sLe^{X/A} moieties to mount shear-resistant, vascular E/P-selectin-mediated adhesion and enhance metastatic potential.

### α2, 6 Sialylation

N-glycan antennae terminated with α2,6 NeuAc moieties (FIG. 10, principally governed by the action of β-galactosyl-α2,6 sialyltransferase (ST6Gal-1), are becoming one of the more critical glycomic features correlated with malignant and metastatic progression. In colon, mammary, ovarian, liver, and pancreatic cancers, α2,6 sialylation can enhance several malignancy-associated activities. Cancer cell α2,6 sialylation can elicit its functional activity on N-glycosylated membrane proteins via a binding moiety (e.g., ligand for siglec-2/CD22) or by imparting optimal stability, membrane organization, or homo/heterophilic interactive capacity. When β1 integrins on cancer cells display ST6Gal-1-synthesized α2,6 sialylated moieties, adhesive and migratory activities and related focal adhesion kinase activities are accentuated. Protection from chemotherapeutics, including EGFR-targeted therapy, and Fasmediated death, promotion of survival pathways, and evasion of hypoxic stress are also boosted in cancer cells via ST6Gal-1-dependent sialylation. Beyond these malignancy-associated traits, tumor-initiating cell activity and expression of stem cell markers have been correlated positively with ST6Gal-1 expression.

### I-Branched Glycans and β1,6 I-Branching Enzyme GCNT2

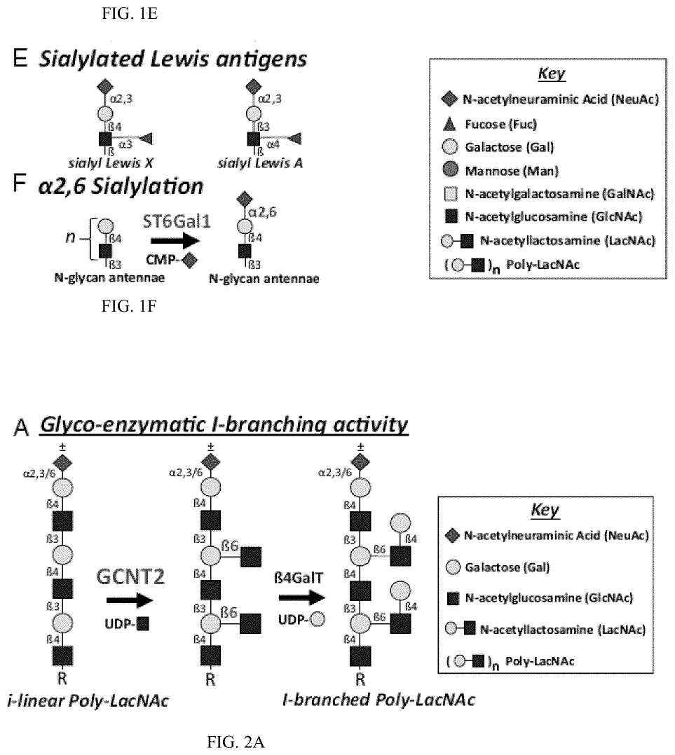

Synthesis of I-branched glycans, Galβ1,4GlcNAc moieties linked in a β1,6 conformation to internal galactose residues on fetal i-antigen [linear poly-LacNAc; (Galβ1,4-GlcNAcβ1,3)n], is chiefly initiated by the developmental I-branching GCNT2 (FIG. 2a). A linear poly-LacNAc synthesized by the repeating action of β3GnTs and β4GalTs provides its internal galactose residues as an acceptor for the β1,6 GlcNAc transferring action of GCNT2 and subsequent ubiquitous β1,4Gal capping activity of β4GalTs. GCNT2 exists as isoforms A, B, and C (also referred to as variants 1, 2, and 3) and governs the conversion of linear poly-LacNAcs commonly expressed on fetal and cord blood cells to I-branched glycans normally found on adult erythrocytes, mucosal epithelia, and cells of the eye and olfactory bulb. Ineffective I-branch conversion has been linked to loss of GCNT2 expression and early-onset congenital cataracts. In cancer, GCNT2/I-branched glycans have been correlated both positively and negatively with cancer progression, regulating malignancy-associated adhesive, migratory, signaling, growth, and metastatic activities as follows.

### Malignant Melanoma.

Melanoma, also known as “malignant melanoma,” is a serious form of skin cancer, and can spread to lymph nodes and internal organs. Melanoma is a malignant tumor of melanocytes which are found predominantly in skin but also in the bowel, oral cavity and the eye. Melanocytes are normally present in skin, being responsible for the production of the dark pigment melanin. Early signs of melanoma include changes to the shape or color of existing moles. The mole may itch, ulcerate or bleed. Metastatic melanoma may cause general symptoms like loss of appetite, nausea, vomiting and fatigue. Treatments include, but are not limited to, surgery, chemotherapy and/or radiation therapy.

GCNT2/I-branching acts as a putative tumor suppressor, inhibiting several malignancy-associated activities in melanoma cells and xenografts. N-glycan antennae on normal epidermal melanocytes almost uniformly display I-branches, whereas primary melanomas variably express I-branched glycans and metastatic melanomas mostly lack I-branches concomitant with depressed GCNT2 expression. Data-mining analysis and immunohistochemical analysis of GCNT2 in clinical primary and metastatic melanoma specimens establish a strong inverse relationship between GCNT2 expression and melanoma metastases, suggesting that GCNT2 expression (or loss thereof) could help serve as a biomarker and predict clinical outcome. Biochemical data show that GCNT2 catalyzes global I-branch synthesis to N-glycans on several classes of membrane proteins expressed by melanoma cells. The presence of GCNT2-synthesized I-branches on growth factor RTKs and α/β integrin chains, such as insulin-like growth factor 1 receptor (IGF1R) and α4-, β1-, and β3-chains, can inhibit IGF1 and extracellular matrix-binding activities and attenuate related downstream signaling and prosurvival factors in melanoma cells.

Mechanistically, the I-branches on normal and malignant melanocytes do not appear to contain sialylated or fucosylated moieties, indicating that the effects of I-branching are likely not through ancillary sialo-fucosylations, but rather as bulky capping moieties causing either direct or indirect steric interference of receptor-ligand interactions.

Further studies show that (i) GCNT2 expression is down-regulated in melanomas, (ii) GCNT2 expression can predict which patients with thick primary melanomas will (or will not) metastasize, and (iii) I-branches antagonize RTK/integrin function in melanoma cells. These can be done using an inducible melanoma mouse model in a wild-type or GCNT2 null background.

### Applications for I-Branched Glycans Controlling Galectin-Binding Activities

In that I-branched glycans—Galß1,4GlcNAc moieties linked in a β1,6 conformation to internal galactose residues on linear poly-LacNAcs—can serve as β-galactoside-binding determinants for galectins and that galectins possess key immunoregulatory and protumorigenic functions, GCNT2/I-branching activity could function as a critical regulator of cancer progression. Because β3GnT extension activity is necessary for linear poly-LacNAc synthesis, β3GnT(s) and GCNT2 could compete to dually regulate the synthesis of linear vs. I-branched poly-LacNAc. GCNT2/I-branching activity, however, appears to serve as an end-stage glycosylation event. GCNT2 and B3GNT2, when coexpressed, have a cooperative relationship, in which I-branched poly-LacNAcs are synthesized from linear poly-LacNAcs and the level of I-branched poly-LacNAcs directly correlates with GCNT2 expression. Such end-stage glycosylation events, akin to α2,6 sialylation and α1,3 fucosylation, often have profound effects on galectin binding activities. Data in studies on GCNT2/I-branching in melanoma progression reveal a potential role for GCNT2/I-branching activity as a native inhibitor of Gal-3 binding activity. As opposed to Gal-1, Gal-3 binds linear poly-LacNAcs on melanoma cells more avidly than to GCNT2-synthesized I-branched glycans, which is consistent with Gal-3's preference for linear poly-LacNAcs on glycan microarrays. Additionally, GCNT2/I-branching activity also blunts Gal-9 ligand activities in numerous melanoma cell lines. Because melanoma progression is directly related to Gal-3 expression in melanoma cells, melanoma-intrinsic GCNT2 action could offset functional activities triggered by Gal-3 binding. That is, in melanoma patients with moderate- to late-stage disease where GCNT2 expression is progressively lost, renewing GCNT2/I-branching activity could potentially antagonize Gal-3-dependent malignant activities and slow melanoma progression.

Coincident with evidence of GCNT2/I-branching antagonizing melanoma galectin ligand activity, intensive glycomic interrogation of human B cell subsets depicts GCNT2 as a major factor controlling Gal-9 binding activity. In contrast to robust binding on naïve and memory B cells, Gal-9 binding to germinal center B cells is markedly less due, in part, to upregulation of GCNT2/I-branching activity. Gal-9, in the absence of I-branched glycans, imposes a regulatory activity via CD45 binding and downstream suppression of B cell receptor signaling and cell activation. Elevated GCNT2/I-branching activity in human B cell lines associates with depressed Gal-3 binding, suggesting that GCNT2 elicits its galectin inhibitory effects across normal and malignant settings.

Collectively, the putative tumor-intrinsic and immunological consequences of GCNT2/I-branching on Gal-3 and Gal-9 function provide opportunities for anticancer therapeutic targeting of GCNT2. Whether tuning galectin-dependent immunoregulation of antitumor immune cells or malignancy-associated activities intrinsic to cancer cells, GCNT2/I-branching provide an attractive therapeutic target to the burgeoning field of cancer immunotherapy.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional teams/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

### EXAMPLES

### Experimental Section

### Materials and Methods

### Immunohistochemistry

Antibodies, concentrations and reagents used for immunohistochemistry are cataloged in Table 1.

TABLE 1
Antibodies and reagents used for flow cytometry,
western blotting, and immunohistochemistry.

| Antibodies/Reagents | Source | Identifier | Concentrations |
|---|---|---|---|
| Biotinylated Phaseolus | Vector | Cat# | 0.5 μg/ml |
| vulgaris Leucoagglutinin | Laboratones | B-1115 | (FACS) |
| (PHA-L) lectin | | | |
| Biotinylated Sambucus | Vector | Cat# | 0.5 μg/ml |
| Nigra Lectin (SNA) | Laboratories | B-1035 | (FACS) |
| Biotinylated | Vector | Cat# | 0.5 μg/ml |
| Maackia Amurensis | Laboatories | B-1265 | (FACS) |
| Lectin II (MAL II) | | | |
| Biotinylated Solanum | Vector | Cat# | 0.5 μg/ml |
| Tuberosum (Potato) | Laboratories | B-1165 | (FACS) |
| Lectin (STA) | | | |
| Biotinylated Lycopersicon | Vector | Cat# | 0.5 μg/ml |
| Esculentum (Tomato) | Laboratories | B-1175 | (FACS) |
| Lectin (LEA) | | | 2 μg.ml |
| | | | (Western) |
| Mouse monoclonal anti- | Abeam | Cat# | 5 μg/ml |
| Ganglioside GD3 | | ab11779 | (FACS) |
| (clone R24) | | | |
| PE anti-human CD221 | Biolegend | Cat# | 1 μg/ml |
| (IGF-1R) Antibody (clone | | 351805 | (FACS) |
| 1H7/CD221) | | | |
| APC anti-human CD29 | Biolegend | Cat# | 0.5 μg/ml |
| (β1 integrin) (clone TS2/16) | | 303008 | (FACS) |
| ARC anti-human CD61 (β3 | Biolegend | Cat# | 1.5 μg/ml |
| integrin) (clone VI-PL2) | | 336411 | (FACS) |
| APC anti-human CD49d | Biolegend | Cat# | 0.5 μg/ml |
| (α4 integrin) (clone 9F10) | | 304307 | (FACS) |
| APC anti-human CD49f | Biolegend | Cat# | 0.1 μg/ml |
| (α6 integrin) (clone GoH3) | | 313615 | (FACS) |
| AF647 anti-human CD51 | Biolegend | Cat# | 1.5 μg/ml |
| (αV integrin) (clone P1F6) | | 920005 | (FACS) |
| APC mouse IgG1, k isotype | Biolegend | Cat# | 0.5 μg/ml |
| ctrl (clone MOPC-21) | | 400120 | (FACS) |
| APC ratIgG2, k isotype | Biolegend | Cat# | 1.5 μg/ml |
| ctrl (clone RTK2758) | | 400511 | (FACS) |
| AF647 mIgG1, k isotype | Biolegend | Cat# | 1.5 μg/ml |
| ctrl (clone MOPC-21) | | 400130 | (FACS) |
| Rabbit monoclonal anti- | Cell Signaling | Cat# | 1:1000 |
| phospho-Tyrosine (clone | Technology | 8954 | (Western) |
| P-Tyr-1000) | | | |
| Rabbit monoclonal anti- | Cell Signaling | Cat# | 1:1000 |
| phospho-IGF-1-Receptor β | Technology | 3024 | (Western) |
| (Tyr1135/1136) | | | |
| (clone 19H7) | | | |
| Rabbit monoclonal anti- | Cell Signaling | Cat# | 1:2000 |
| phospho-AKT (Ser473) | Technology | 4060 | (Western) |
| (clone D9E) | | | |
| Rabbit monoclonal | Cell Signaling | Cat# | 1:1000 |
| anti-phospho-AKT | Technology | 13038 | (Western) |
| (Thr308) (clone D25E6) | | | |
| Rabbit monoclonal | Cell Signaling | Cat# | 1:1000 |
| anti-IGF-1 Receptor β | Technology | 9750 | (Western) |
| (clone D23H3) | | | |
| Mouse monoclonal | Cell Signaling | Cat# | 1:2000 |
| anti-AKT (pan) | Technology | 2920 | (Western) |
| (clone 40D4) | | | |
| Rabbit polyclonal anti- | Cell Signaling | Cat# | 1:1000 |
| phospho-FAK (Tyr576/577) | Technology | 3281 | (Western) |
| Rabbit monoclonal anti- | Cell Signaling | Cat# | 1:2000 |
| phospho-p44/42 MAPK | Technology | 4370 | (Western) |
| (Erk1/2) (Thr202/Tyr204) | | | |
| (clone D13.14.4E) | | | |
| Rabbit monoclorial anti- | Cell Signaling | Cat# | 1:1000 |
| FAK (clone D2R2E) | Technology | 13009 | (Western) |

Sections of archival FFPE human normal skin, nevi or melanoma tissue microarray (TMA) were kindly provided by Dr. Richard Scolyer (Melanoma Institute of Australia). Sections were deparaffinized in xylene and subsequently rehydrated with 100%, 95% and 75% ethanol and deionized water. Sections were then placed in antigen retrieval solution and boiled at 100° C. for 20 min. Sections were then stained with a 1:500 dilution of GCNT2 antibody (Sigma-Aldrich) for 30 min at 37° C. GCNT2 primary antibody was detected using the Leica Bond Polymer Refine Detection Kit (Leica #DS9800), the polymer-horse radish peroxidase secondary antibody is incubated for 15 min at room temperature. All sections were counterstained in hematoxylin. Images were acquired using a Nikon eclipse Ti microscope and a Nikon FDX-35 digital camera.

For TMA “grade” scoring, individual cores in GCNT2-stained TMA cores were first excluded if melanocyte/melanoma cells were absent or tissue quality deemed unsuitable by pathologist, melanoma cells (as identified/confirmed by a pathologist) were graded as follows: random fields in nevi/melanomas were analyzed [>100 cells total for all specimens; semi-quantitatively graded as 0, 1 (1-25% cells positive); 2 (25-50% cells positive); 3 (50-75% cells positive); 4 (75-100% cells positive). For TMA “intensity” scoring, random fields in nevi/melanomas were analyzed [>100 cells total for all specimens; semi-quantitatively graded as 0 (No staining), 1 (Faint staining); 2 (Moderate staining); 3 (Dark staining). Clinical outcome data of Alive No Such Recurrence, Alive with Melanoma, Dead with Melanoma was then matched to the corresponding stained specimens and analyzed for statistical significance via Cochran-Armitage Trend Test (p-value<0.05). All IHC scoring was performed in a blinded manner.

### Statistical Analysis

Statistical analyses were performed using Prism 7.0 software (GraphPad). For tests involving two groups, testing was carried out using unpaired two-tailed Student's t test. When more than two groups were compared, a one-way analysis of variance (ANOVA) followed by Dunnett's multiple comparisons tests were performed. A two-way ANOVA followed by either Bonferroni's (two groups) or Dunnett's (more than two groups) multiple comparisons tests were used in cases where more than two groups were compared with repeated measures (i.e., in vivo tumor growth). For correlation of I-branch glycan expression and GCNT2 gene expression, linear regression was used. Statistical analysis for GlycoV4 microarray were done with limma, and Benjamini-Hochberg correction was used to adjust the p values. Only genes with absolute fold change >2 and adjusted p value <0.05 were considered differentially expressed. Based on statistical significance assessments in prior published data sets on the role of glycomics and cancer cell biology, we performed all in vitro and in vivo assessments a minimum of three times, unless otherwise noted. Data are presented as the means±SEM. P values <0.05 was were considered significant.

### Example 1—GCNT2 Level Affects Survival of Stage 2/3 Melanoma Patients

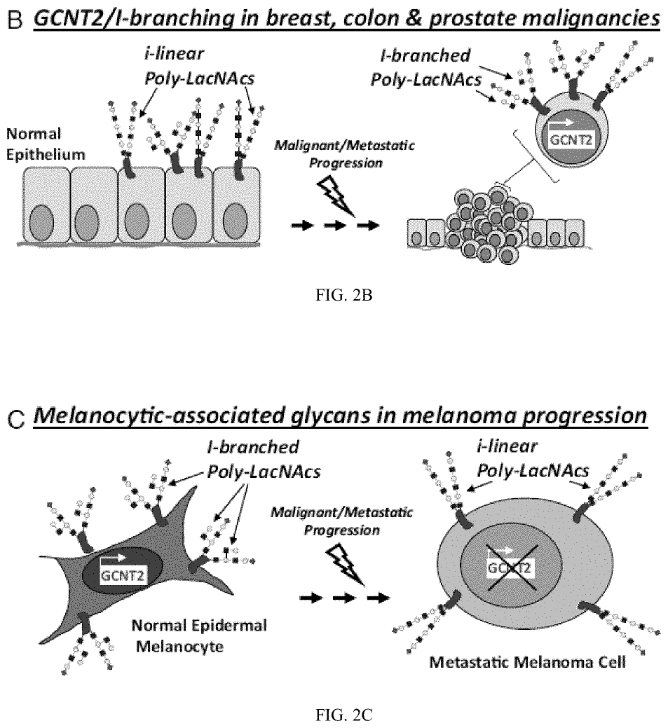

GCNT2 is down-regulated in melanomas. To determine the relationship between GCNT2 and melanoma-related death, samples from 64 stage 2/3 melanoma patients were stained specifically for GCNT2. Majority of the patients died from melanoma show none or light staining intensity of GCNTs, indicating none or little expression of GCNT2 in the samples of these patients (FIG. 3). Patients showing moderate or dark staining intensity of GCNT2 have a significant reduced death rate (FIG. 3). These results show that increasing the expression level of GCNT2 in melanoma patients may be a strategy for treating melanomas, slowing the progression of melanomas and increasing the survival rate of melanoma patients.

### Example 2—GCNT2 Expression Level in IC Therapy Responding and Non-Responding Patients

PD-1 immune checkpoint blockade therapy has been used against human malignancies. However, patients with advanced metastatic melanoma show a high rate of innate resistance (60-70%) to anti-PD-1 agents. In melanoma, tumor-specific T cells are the primary mechanistic basis of anti-PD-1 therapy. The extent of pretreatment and especially treatment-induced intra-tumoral T cell infiltration correlates with clinical responses.

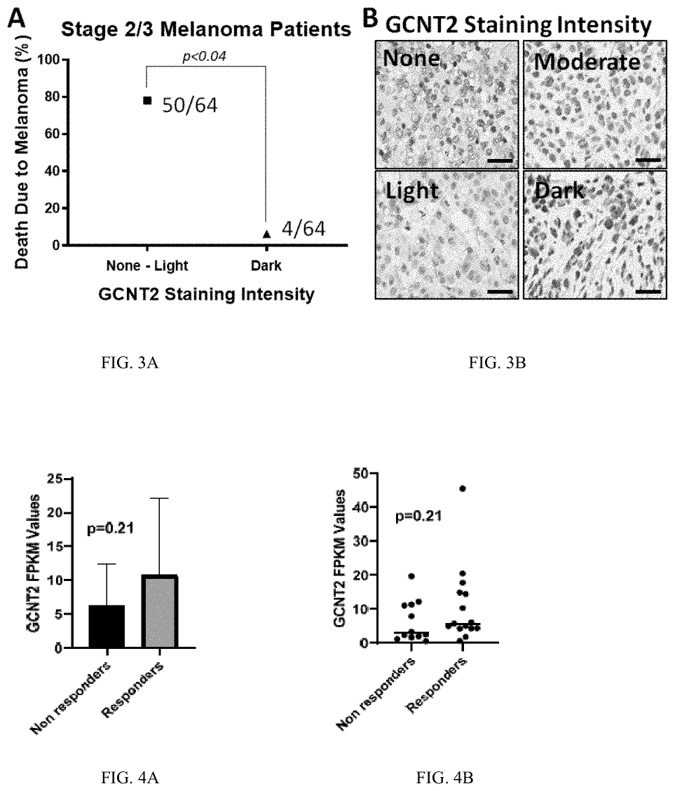

38 tumor samples and their respective normal tissues are collected as described in Hugo et al. Expressions of GCNT2 are normalized in fragments per kilobase of transcripts per million mapped reads (FPKM). As shown in FIG. 4, patients with higher levels of GCNT2 corresponded with Immune Checkpoint Inhibitor (ICI) therapy Responders, whereas patients with lower levels of GCNT2 corresponded with ICI Nonresponders to ICI therapy. Thus, higher GCNT2 expression in patients corresponds with a better outcome or response to immune checkpoint therapy.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

We claim:

1. A method for treating melanoma in a subject, comprising administering to the subject a pharmaceutical composition comprising 1) a nucleic acid sequence that encodes GCNT2 or a nucleic acid sequence sharing at least 95% identity with the nucleic acid sequence that encodes GCNT2, 2) an amino acid sequence of GCNT2 or an amino acid sequence sharing at least 95% identity with the amino acid sequence of GCNT2, and/or 3) a vector comprising a nucleic acid sequence that encodes GCNT2 or a nucleic acid sequence sharing at least 95% identity with the nucleic acid sequence that encodes GCNT2.

2. The method of claim 1, the administration being local, topical, or intravenous administration.

3. The method of claim 1, the subject being a human.

4. The method of claim 1, the melanoma being stage II, III or IV melanoma.

5. The method of claim 1, the nucleic acid sequence of GCNT 2 being SEQ ID NO: 1.

6. The method of claim 1, the amino acid sequence of GCNT2 being SEQ ID NO: 2.

7. The method of claim 1, the melanoma being immune checkpoint inhibitor (ICI) therapy-resistant melanoma.

8. The method of claim 1, the subject having been treated with an ICI therapy.

9. The method of claim 7, the ICI being selected from antibodies of cytotoxic T-lymphocyte antigen 4 (CTLA-4), programmed cell death protein 1 (PD-1), PD-1 ligand (PDL-1), T-cell immunoglobulin and mucin domain-containing protein 3 (TIM-3), killer cell immunoglobulin-like receptor (KIR), lymphocyte activation gene-3 (LAG-3), V-domain immunoglobulin suppressor of T cell activation (VISTA), and B and T lymphocyte attenuator (BTLA).

10. The method of claim 1, the ICI therapy being anti-PD-1 therapy.

11. A method for slowing the growth of melanoma cells, the method comprising contacting the melanoma cells with a composition comprising 1) a nucleic acid sequence that encodes GCNT2, 2) an amino acid sequence of GCNT2, and/or 3) a vector comprising a nucleic acid sequence that encodes GCNT2.

12. The method of claim 11, the melanoma cells having reduced expression level of GCNT2 prior to contacting the composition.

13. The method of claim 11, the melanoma cells being resistant to one or more ICIs.

14. The method of claim 13, the ICI being selected from antibodies of cytotoxic T-lymphocyte antigen 4 (CTLA-4), programmed cell death protein 1 (PD-1), PD-1 ligand (PDL-1), T-cell immunoglobulin and mucin domain-containing protein 3 (TIM-3), killer cell immunoglobulin-like receptor (KIR), lymphocyte activation gene-3 (LAG-3), V-domain immunoglobulin suppressor of T cell activation (VISTA), and B and T lymphocyte attenuator (BTLA).

15. A method for increasing the sensitivity of a subject having melanoma to an ICI therapy, the method comprising administering to the subject a pharmaceutical composition comprising 1) a nucleic acid sequence that encodes GCNT2, 2) an amino acid sequence of GCNT2, and/or 3) a vector comprising a nucleic acid sequence that encodes GCNT2.

16. The method of claim 15, the subject having been treated with an ICI therapy.

17. The method of claim 15, the melanoma being ICI therapy-resistant melanoma.

18. The method of claim 15, the administration being local, topical, or intravenous administration.

19. The method of claim 15, the composition being administered prior to, simultaneously with or, after the administration of the ICI therapy.

20. The method of claim 16, the ICI therapy being anti-PD-1 therapy.
